(19) United States
(12) Patent Application Publication
(10) Pub. No.: US 2023/0075937 A1
(43) Pub. Date: Mar. 9, 2023

(54) METHODS FOR STRENGTHENING AND REPAIRING HAIR

(71) Applicant: Living Proof, Inc., Boston, MA (US)

(72) Inventors: Zhaoxia Ji, Natick, MA (US); Nawodi Abeyrathna, Boston, MA (US); Harold Bryant, Boston, MA (US); Ronald P. McLaughlin, Boston, MA (US)

(21) Appl. No.: 17/899,978

(22) Filed: Aug. 31, 2022

Related U.S. Application Data
(60) Provisional application No. 63/238,350, filed on Aug. 30, 2021.

(51) Int. Cl. A61K 8/55; A61K 8/49; A61Q 5/00

(52) U.S. Cl. CPC A61K 8/55; A61K 8/498; A61Q 5/002

(57) ABSTRACT

Provided are method for using phytic acid (PA), alone or in combination with glucono-delta-lactone (GL) to strengthen and/or repair hair.

### RELATED APPLICATIONS

This application claims the benefit of priority to U.S. Provisional Application No. 63/238350, filed Aug. 20, 2021, the entire contents of which are incorporated herein by reference.

### BACKGROUND

General hair practices of consumers, such as regular washing cycles, mechanical and chemical processing, thermal styling, and other environmental factors can lead to various types of hair damage, both on the hair surface and inside the cortex. To address these problems and repair hair damage, it is important to develop effective technologies that not only provide hair surface repair but also internal structural strengthening.

### SUMMARY

Treating damaged hair with phytic acid (PA), alone or in combination with glucono-delta-lactone (GL), has now been found to result in significant improvements in the thermal and/or mechanical properties of the hair. See e.g., FIGS. 1, 2A, 2B, and 3, where treatment of the hair with 2 wt % phytic acid and a combination of 2 wt % phytic acid and 2 wt % glucono-delta-lactone was found to improve the denaturation temperature (Td), Young's modulus, and break stress compared to untreated hair. Further results and data are provided in the Exemplification section below.

Additionally, it was also found using PA alone, or in combination with GL, improved surface hydrophobicity and reduced protein loss in damaged hair. See e.g., FIGS. 4 and 5, where treatment of hair with 2 wt % phytic acid and a combination of 2 wt % phytic acid and 2 wt % glucono-delta-lactone was found to improve the hydrophobicity of the hair and minimize protein loss.

In one aspect, therefore, provided herein is the use of phytic acid (PA), alone or in combination with glucono-delta-lactone (GL), for repairing and/or strengthening human hair.

Through future studies, it was also identified that PA had protective effects against heat damage or chemical treatment to the hair. See e.g., FIG. 31 where hair treated with PA after bleach showed immediate improvement in Td (3° C.), even beyond the virgin state, and remained above the virgin level over 12 washing cycles and even after additional bleaching. See also FIG. 32 where PA treated hair had the same Td as virgin hair even after 3 heating cycles.

In one aspect, therefore, provided herein is the use of phytic acid (PA), alone or in combination with glucono-delta-lactone (GL), for protecting hair e.g., from heat or chemical treatments.

### BRIEF DESCRIPTION OF THE DRAWINGS

FIG. 1 shows denaturation temperatures of untreated virgin hair 3× bleached and 3× bleached hair treated with a 2 wt % aqueous phytic acid solution and an aqueous solution comprising 2 wt % phytic acid and 2 wt % glucono-delta-lactone.

FIG. 2A shows the percent of Young's modulus (YM) change upon the treatment with a 2 wt % aqueous phytic acid solution and an aqueous solution comprising 2 wt % phytic acid and 2 wt % glucono-delta-lactone followed by heat styling (blow dry or flatiron) on bleached hair.

FIG. 2B shows the percent break stress change upon the treatment with a 2 wt % aqueous phytic acid solution and an aqueous solution comprising 2 wt % phytic acid and 2 wt % glucono-delta-lactone followed by heat styling (blow dry or flatiron) on bleached hair.

FIG. 3 shows the percent of Young's modulus (YM) and break stress change upon the treatment with 2 wt % aqueous phytic acid solution on an over bleached hair model.

FIG. 4 shows the water contact angle change upon the treatment with a 2 wt % aqueous phytic acid solution and an aqueous solution comprising 2 wt % phytic acid and 2 wt % glucono-delta-lactone w.r.t. 3× bleached and that of an aqueous solution comprising 2 wt % phytic acid and 2 wt % glucono-delta-lactone treated hair w.r.t. 2 wt % phytic acid treated hair.

FIG. 5 shows the protein loss concentration change on 3× bleached hair upon the treatment with a 2 wt % aqueous phytic acid solution and a combination of an aqueous solution comprising 2 wt % phytic acid and 2 wt % glucono-delta-lactone compared to 3× bleached hair.

FIG. 6 shows the percent area change at a 65% relative humidity for 30 min, for 3× bleached hair treated with a 2 wt % aqueous phytic acid solution and an aqueous solution comprising 2 wt % phytic acid and 2 wt % glucono-delta-lactone followed by heat styling compared to that of untreated 3× bleached hair, after 1, 3 and 7 washing cycles.

FIG. 7A shows the percent of Young's modulus (YM) change upon the treatment with a 2 wt % aqueous phytic acid solution and an aqueous solution comprising 2 wt % phytic acid and 2 wt % glucono-delta-lactone from pH 2 to 5, followed by heat styling (flatiron) on bleached hair.

FIG. 7B shows the percent break stress change upon the treatment with a 2 wt % aqueous phytic acid solution and an aqueous solution comprising 2 wt % phytic acid and 2 wt % glucono-delta-lactone from pH 2 to 5, followed by heat styling (flatiron) on bleached hair.

FIG. 8A shows the percent of Young's modulus (YM) change upon the treatment with phytic acid solutions at different concentrations, followed by heat styling (flatiron) on bleached hair.

FIG. 8B shows the percent break stress change upon the treatment with phytic acid solutions at different concentrations, followed by heat styling (flatiron) on bleached hair.

FIG. 9 shows the denaturation temperature of hair samples treated with phytic acid solutions at different concentrations, followed by heat styling (flatiron) as compared to untreated virgin and bleached hair.

FIG. 10 shows the denaturation temperature of reduced hair sample post-treated with a 2 wt % aqueous phytic acid solution followed by blow dry as compared to untreated (virgin) and reduced hair.

FIG. 11 shows the water contact angle change upon the treatment with a 2 wt % aqueous phytic acid solution and an aqueous solution comprising 2 wt % phytic acid and 2 wt % glucono-delta-lactone w.r.t. ATG reduced hair and that of an aqueous solution comprising 2 wt % phytic acid and 2 wt % glucono-delta-lactone solution treated hair w.r.t. 2 wt % aqueous phytic acid solution treated hair.

FIG. 12 shows the protein loss concentration change upon the treatment with a 2 wt % aqueous phytic acid solution and a combination of an aqueous solution comprising 2 wt % phytic acid and 2 wt % glucono-delta-lactone compared to ATG reduced hair.

FIG. 13 shows the denaturation temperature of hair treated with color followed by a 2 wt % aqueous phytic acid solution and a combination of an aqueous solution comprising 2 wt % phytic acid and 2 wt % glucono-delta-lactone post-treatment solutions with heat styling (blow dry) as compared to untreated and colored hair without post treatment.

FIG. 14A shows the percent of Young's modulus change upon the post-treatment with an aqueous solution comprising 2 wt % phytic acid and 2 wt % glucono-delta-lactone followed by heat styling (blow dry) on colored hair compared to color treatment alone.

FIG. 14B shows the percent break stress change upon the post-treatment with an aqueous solution comprising 2 wt % phytic acid and 2 wt % glucono-delta-lactone followed by heat styling (blow dry) on colored hair compared to color treatment alone.

FIG. 15 shows the water contact angle change upon the treatment with a 2 wt % aqueous phytic acid solution and an aqueous solution comprising 2 wt % phytic acid and 2 wt % glucono-delta-lactone w.r.t. damaged hair (colored) and that of an aqueous solution comprising 2 wt % phytic acid and 2 wt % glucono-delta-lactone treated hair w.r.t. 2 wt % aqueous phytic acid solution treated hair.

FIG. 16A shows the percent of Young's modulus (YM) change upon the treatment with phytic acid containing base formulation, followed by heat styling (flatiron) on bleached hair.

FIG. 16B shows the percent of break stress change upon the treatment with phytic acid containing base formulation, followed by heat styling (flatiron) on bleached hair.

FIG. 17 shows the percent relative diameter swelling of hair types (virgin hair, bleached hair and bleached hair treated with 2% PA followed by blow dry) at various percent relative humidity.

FIG. 18 shows the smooth fracture pattern distribution for virgin brown hair, 3× bleached hair before and after PA treatment.

FIG. 19 shows denaturation temperature of heat damaged hair followed by PA treatment as compared to untreated virgin and heat damaged hair.

FIG. 20 shows denaturation temperature of hair samples subjected to ₃ heating cycles with 2% PA treatment as compared to untreated virgin and virgin hair treated with 3 heating cycles.

FIG. 21 shows denaturation temperature of 3× bleached hair treated with PA formulation followed by blow dry as compared to untreated virgin hair, bleached hair and 2 bond builder comparators.

FIG. 22 shows the percent of Young's modulus (YM) upon the treatment with PA formulation followed by heat styling (blow dry) on 3× bleached hair compared to 2 bond building comparators.

FIG. 23 shows the percent break stress change upon the treatment with PA formulation followed by heat styling (blow dry) on 3× bleached hair compared to 2 bond building comparators.

FIG. 24 shows denaturation temperature of heat damaged hair treated with PA formulation followed by heat styling (blow dry) as compared to untreated virgin and heat damaged hair.

FIG. 25 shows the percent of Young's modulus (YM) change upon the treatment with PA formulation on 3× bleached hair followed by different drying and styling methods.

FIG. 26 shows the percent break stress change upon the treatment with PA formulation on 3× bleached hair followed by different drying and styling methods.

FIG. 27 is a comparison of the count of broken hair fragments at different combing cycle intervals up to 10,000 combing cycles, between virgin, bleached, PA formulation and 2 comparator formulations without PA.

FIG. 28A shows the percent of smooth fracture patterns for virgin brown hair, 3× bleached hair before and after PA treatment.

FIG. 28B shows SEM images of a pair of smooth fracture ends of PA formulation treated bleached hair.

FIG. 29 shows the water contact angle change upon the treatment with PA formulation compared to virgin and 3× bleached hair.

FIG. 30 shows protein loss concentration change on 3× bleached hair upon the treatment with PA formulation compared to virgin and 3× bleached hair.

FIG. 31 shows the change in hair denaturation temperature after bleaching and over 12 shampoo and conditioner washing cycles with or without PA formulation treatments.

FIG. 32 shows denaturation temperature of hair samples subjected to 3 heating cycles with PA formulation treatment as compared to untreated virgin and Virgin hair treated with 3 heating cycles.

### DETAILED DESCRIPTION

In one aspect, provided are methods for strengthening and/or repairing human hair comprising applying to the hair an effective amount of phytic acid. In another aspect, provided are methods for strengthening and/or repairing damaged human hair comprising applying to the damaged hair an effective amount of phytic acid. In another aspect, provided are methods for strengthening and/or repairing human hair comprising applying to hair an effective amount of phytic acid, wherein the human hair has been bleached prior to applying the phytic acid. In another aspect, provided are methods for strengthening and/or repairing human hair comprising applying to hair an effective amount of phytic acid, wherein disulfide bonds of the human hair have been reduced (e.g., with a reducing agent such as ammonium thioglycolate) prior to applying the phytic acid. In another aspect, provided are methods for strengthening and/or repairing human hair comprising applying to hair an effective amount of phytic acid, wherein the human hair has been color treated prior to applying the phytic acid.

In one aspect, provided are methods for protecting hair from heat or chemical treatments comprising applying to the hair an effective amount of phytic acid. In one aspect, provided are methods for protecting hair from chemical treatments comprising applying to the hair an effective amount of phytic acid, wherein the phytic acid is applied prior to chemical treating e.g., bleaching. In one aspect, provided are methods for protecting hair from heat comprising applying to the hair an effective amount of phytic acid, wherein the phytic acid is applied prior to heating the hair.

In one aspect, provided are methods for strengthening and/or repairing human hair comprising applying to the hair an effective amount of phytic acid and an effective amount of glucono-delta-lactone. In another aspect, provided are methods for strengthening and/or repairing damaged human hair comprising applying to the damaged hair an effective amount of phytic acid and an effective amount of glucono-delta-lactone. In another aspect, provided are methods for strengthening and/or repairing human hair comprising applying to hair an effective amount of phytic acid and an effective amount of glucono-delta-lactone, wherein the human hair has been bleached prior to applying the phytic acid and glucono-delta-lactone. In another aspect, provided are methods for strengthening and/or repairing human hair comprising applying to hair an effective amount of phytic acid and an effective amount of glucono-delta-lactone, wherein disulfide bonds of the human hair have been reduced (e.g., with a reducing agent such as ammonium thioglycolate) prior to applying the phytic acid and glucono-delta-lactone. In another aspect, provided are methods for strengthening and/or repairing human hair comprising applying to hair an effective amount of phytic acid and an effective amount of glucono-delta-lactone, wherein the human hair has been color treated prior to applying the phytic acid and glucono-delta-lactone.

In one aspect, the methods described herein further comprise heating the hair after applying phytic acid, alone or in combination with glucono-delta-lactone. In certain aspects, said heating occurs a temperature ranging from 40° C. to 232° C. In other aspects, said heating occurs a temperature ranging from 40° C. to 232° C. for a period of time ranging from 5 seconds to 30 minutes.

As used herein, “strengthening” hair refers to an improvement in one or more thermal and/or mechanical properties of the hair resulting from treatment with phytic acid or a combination of phytic acid and glucono-delta-lactone. Thermal and mechanical properties of the hair include, but are not limited to, denaturation temperature, Young modulus, break extension, break stress, hair breakage, and fiber fatigue.

As used herein, “repairing” hair refers to an improvement in hair structural integrity and/or surface quality. Such improvements include, but are not limited to, reduced swelling, reduced protein loss, increased surface hydrophobicity, and improved humidity resistance.

Phytic acid, also known as inositol hexakisphosphate (IP6) or inositol polyphosphate, has the chemical structure:

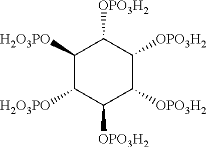

As used herein, an “effective amount of phytic acid” refers to an amount of phytic acid that is sufficient to strengthen hair. Phytic acid may be applied directly to the hair or as a component in a cosmetically acceptable composition. In one aspect, an effective amount of phytic acid comprises phytic acid as part of an aqueous solution. In one aspect, an effective amount of phytic acid comprises applying at least about a 0.1 wt % aqueous phytic acid solution to the hair such as a 1.5 g hair tress. In another aspect, an effective amount of phytic acid comprises applying about 0.1 wt % to about 50 wt % aqueous phytic acid solution to the hair such as a 1.5 g hair tress. In another aspect, an effective amount of phytic acid comprises applying about 0.5 wt % to about 50 wt % aqueous phytic acid solution to the hair such as a 1.5 g hair tress. In another aspect, an effective amount of phytic acid comprises applying at least about a 2 wt % aqueous phytic acid solution to the hair. In another aspect, an effective amount of phytic acid comprises applying about 2 wt % to about 50 wt % aqueous phytic acid solution to the hair such as a 1.5 g hair tress. In another aspect, an effective amount of phytic acid comprises applying about 2 wt % to about 20 wt % aqueous phytic acid solution to the hair such as a 1.5 g hair tress. In another aspect, an effective amount of phytic acid comprises applying about 2 wt % to about 15 wt % aqueous phytic acid solution to the hair such as a 1.5 g hair tress. In another aspect, an effective amount of phytic acid comprises applying about 2 wt % to about 10 wt % aqueous phytic acid solution to the hair such as a 1.5 g hair tress. In another aspect, an effective amount of phytic acid comprises applying about 0.5 wt % to about 1 wt % aqueous phytic acid solution to the hair such as a 1.5 g hair tress. In another aspect, an effective amount of phytic acid comprises applying about 1 wt % to about 3 wt % aqueous phytic acid solution to the hair such as a 1.5 g hair tress. In another aspect, an effective amount of phytic acid comprises applying about 2 wt %, about 5 wt %, about 10 wt %, about 15 wt %, or about 20 wt % aqueous phytic acid solution to the hair such as a 1.5 g hair tress. In another aspect, an effective amount of phytic acid comprises applying about 0.5 wt %, 2 wt %, about 5 wt %, about 10 wt %, about 15 wt %, or about 20 wt % aqueous phytic acid solution to the hair such as a 1.5 g hair tress. In another aspect, an effective amount of phytic acid comprises applying about 0.75 wt % or about 2 wt %, aqueous phytic acid solution to the hair such as a 1.5 g hair tress. In another aspect, an effective amount of phytic acid comprises applying about 2 wt % aqueous phytic acid solution to the hair such as a 1.5 g hair tress. In another aspect, an effective amount of phytic acid comprises applying about 0.75 wt % aqueous phytic acid solution to the hair such as a 1.5 g hair tress.

When a range is described, it is to be understood that all ranges and individual data points, including the end points are intended. For example, about 1 wt % to about 3 wt % covers about 1 wt % to about 2 wt %, about 1.5 wt % to about 2 wt %, about 2 wt % to about 3, about 2.5 wt % to about 3 wt %, about 1 wt %, about 1.1 wt %, about 1.2 wt %, about 1.3 wt %, about 1.4 wt %, about 1.5 wt %, . . . about 2 wt %, and all other ranges and data points in between.

In one aspect, an effective amount of phytic acid comprises applying about 0.2 grams to about 2.0 grams of about 0.1 wt % to about 50 wt % aqueous phytic acid solution to the hair such as a 1.5 g hair tress. In another aspect, an effective amount of phytic acid comprises applying about 0.2 grams to about 1.5 grams of about 0.1 wt % to about 50 wt % aqueous phytic acid solution to the hair such as a 1.5 g hair tress. In another aspect, an effective amount of phytic acid comprises applying about 0.2 grams to about 1.0 grams of about 0.1 wt % to about 50 wt % aqueous phytic acid solution to the hair such as a 1.5 g hair tress. In another aspect, an effective amount of phytic acid comprises applying about 0.2 grams to about 2.0 grams of at least about a 0.1 wt % aqueous phytic acid solution to the hair such as a 1.5 g hair tress. In another aspect, an effective amount of phytic acid comprises applying about 0.2 grams to about 1.5 grams of at least about a 0.1 wt % aqueous phytic acid solution to the hair such as a 1.5 g hair tress. In another aspect, an effective amount of phytic acid comprises applying about 0.2 grams to about 1.0 grams of at least about a 0.1 wt % aqueous phytic acid solution to the hair such as a 1.5 g hair tress. In another aspect, an effective amount of phytic acid comprises applying about 0.2 grams to about 2.0 grams of at least about a 2 wt % aqueous phytic acid solution to the hair such as a 1.5 g hair tress. In another aspect, an effective amount of phytic acid comprises applying about 0.2 grams to about 2.0 grams of at least about a 1.5 wt % aqueous phytic acid solution to the hair such as a 1.5 g hair tress. In another aspect, an effective amount of phytic acid comprises applying about 0.2 grams to about 2.0 grams of at least about a 1 wt % aqueous phytic acid solution to the hair such as a 1.5 g hair tress. In another aspect, an effective amount of phytic acid comprises applying about 0.2 grams to about 2.0 grams of about 2 wt % to about 20 wt % aqueous phytic acid solution to the hair such as a 1.5 g hair tress. In another aspect, an effective amount of phytic acid comprises applying about 0.2 grams to about 1.5 grams of about 2 wt % to about 20 wt % aqueous phytic acid solution to the hair such as a 1.5 g hair tress. In another aspect, an effective amount of phytic acid comprises applying about 0.2 grams to about 1.0 grams of about 2 wt % to about 20 wt % aqueous phytic acid solution to the hair such as a 1.5 g hair tress.

In another aspect, an effective amount of phytic acid comprises applying about 0.2 grams to about 2.0 grams of about 2 wt % to about 15 wt % aqueous phytic acid solution to the hair such as a 1.5 g hair tress. In another aspect, an effective amount of phytic acid comprises applying about 0.2 grams to about 1.5 grams of about 2 wt % to about 15 wt % aqueous phytic acid solution to the hair such as a 1.5 g hair tress. In another aspect, an effective amount of phytic acid comprises applying about 0.2 grams to about 1.0 grams of about 2 wt % to about 15 wt % aqueous phytic acid solution to the hair such as a 1.5 g hair tress. In another aspect, an effective amount of phytic acid comprises applying about 0.2 grams to about 2.0 grams of about 2 wt % to about 10 wt % aqueous phytic acid solution to the hair such as a 1.5 g hair tress. In another aspect, an effective amount of phytic acid comprises applying about 0.2 grams to about 1.5 grams of about 2 wt % to about 10 wt % aqueous phytic acid solution to the hair such as a 1.5 g hair tress. In another aspect, an effective amount of phytic acid comprises applying about 0.2 grams to about 1.0 grams of about 2 wt % to about 10 wt % aqueous phytic acid solution to the hair such as a 1.5 g hair tress. In another aspect, an effective amount of phytic acid comprises applying about 0.2 grams to about 2.0 grams of about 2 wt %, about 5 wt %, about 10 wt %, about 15 wt %, or about 20 wt % aqueous phytic acid solution to the hair such as a 1.5 g hair tress. In another aspect, an effective amount of phytic acid comprises applying about 0.2 grams to about 1.5 grams of about 2 wt %, about 5 wt %, about 10 wt %, about 15 wt %, or about 20 wt % aqueous phytic acid solution to the hair such as a 1.5 g hair tress. In another aspect, an effective amount of phytic acid comprises applying about 0.2 grams to about 1.0 grams of about 2 wt %, about 5 wt %, about 10 wt %, about 15 wt %, or about 20 wt % aqueous phytic acid solution to the hair such as a 1.5 g hair tress.

It will be appreciated that depending upon the pH of the medium, phytic acid may exist as an ionized, non-ionized, and partially ionized form, each of which are encompassed by the term “phytic acid.”

In one aspect, when used as component in a cosmetically acceptable composition, an effective amount of phytic acid ranges from about 0.1 wt % to about 50 wt % based on the total weight of the cosmetically acceptable composition. In another aspect, when used as component in a cosmetically acceptable composition, an effective amount of phytic acid ranges from about 0.1 wt % to about 20 wt % based on the total weight of the cosmetically acceptable composition. In another aspect, when used as component in a cosmetically acceptable composition, an effective amount of phytic acid ranges from about 0.1 wt % to about 15 wt % based on the total weight of the cosmetically acceptable composition. In another aspect, when used as component in a cosmetically acceptable composition, an effective amount of phytic acid ranges from about 2 wt % to about 50 wt % based on the total weight of the cosmetically acceptable composition. In another aspect, when used as component in a cosmetically acceptable composition, an effective amount of phytic acid ranges from about 2 wt % to about 20 wt % based on the total weight of the cosmetically acceptable composition. In another aspect, when used as component in a cosmetically acceptable composition, an effective amount of phytic acid ranges from about 2 wt % to about 15 wt % based on the total weight of the cosmetically acceptable composition. In another aspect, when used as component in a cosmetically acceptable composition, an effective amount of phytic acid is about 2 wt %, about 5 wt %, or about 15 wt % based on the total weight of the cosmetically acceptable composition. In another aspect, when used as component in a cosmetically acceptable composition, an effective amount of phytic acid is about 2 wt % based on the total weight of the cosmetically acceptable composition.

As used herein, an “glucono-delta-lactone”, also known as gluconolactone, having the structure:

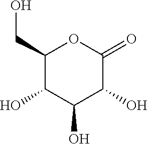 [SMILES: O=C1O[C@H](CO)[C@@H](O)[C@H](O)[C@H]1O]

refers to an amount of glucono-delta-lactone that is sufficient to maintain or improve hair strength when used in combination with phytic acid. Glucono-delta-lactone may be applied directly to the hair or as a component in a cosmetically acceptable composition. When hair is treated with a combination of phytic acid and glucono-delta-lactone, it will be understood that the glucono-delta-lactone may be applied shortly before, concurrently with, or shortly after applying phytic acid. In one aspect, however, glucono-delta-lactone and phytic acid are mixed together and applied to the hair at the same time. In one aspect, an effective amount of glucono-delta-lactone comprises applying at least about 0.1 wt % aqueous glucono-delta-lactone solution to the hair such as a 1.5 g hair tress. In another aspect, an effective amount of glucono-delta-lactone comprises applying about 0.1 wt % to about 50 wt % aqueous glucono-delta-lactone solution to the hair such as a 1.5 g hair tress. In another aspect, an effective amount of glucono-delta-lactone comprises applying about 2 wt % aqueous glucono-delta-lactone solution to the hair such as a 1.5 g hair tress. In another aspect, an effective amount of glucono-delta-lactone comprises applying about 2 wt % to about 50 wt % aqueous glucono-delta-lactone solution to the hair. In another aspect, an effective amount of glucono-delta-lactone comprises applying about 2 wt % to about 20 wt % aqueous glucono-delta-lactone solution to the hair such as a 1.5 g hair tress. In another aspect, an effective amount of glucono-delta-lactone comprises applying about 2 wt % to about 15 wt % aqueous glucono-delta-lactone solution to the hair such as a 1.5 g hair tress. In another aspect, an effective amount of glucono-delta-lactone comprises applying about 2 wt % to about 10 wt % aqueous glucono-delta-lactone solution to the hair. In another aspect, an effective amount of glucono-delta-lactone comprises applying about 2 wt % aqueous glucono-delta-lactone solution to the hair such as a 1.5 g hair tress.

In one aspect, an effective amount of glucono-delta-lactone comprises applying about 0.2 grams to about 2.0 grams of about 0.1 wt % to about 50 wt % aqueous glucono-delta-lactone solution to the hair such as a 1.5 g hair tress. In another aspect, an effective amount of glucono-delta-lactone comprises applying about 0.2 grams to about 1.5 grams of about 0.1 wt % to about 50 wt % aqueous glucono-delta-lactone solution to the hair such as a 1.5 g hair tress. In another aspect, an effective amount of glucono-delta-lactone comprises applying about 0.2 grams to about 1.0 grams of about 0.1 wt % to about 50 wt % aqueous glucono-delta-lactone solution to the hair such as a 1.5 g hair tress. In another aspect, an effective amount of glucono-delta-lactone comprises applying about 0.2 grams to about 2.0 grams of at least about a 0.1 wt % aqueous glucono-delta-lactone solution to the hair such as a 1.5 g hair tress. In another aspect, an effective amount of glucono-delta-lactone comprises applying about 0.2 grams to about 1.5 grams of at least about a 0.1 wt % aqueous glucono-delta-lactone solution to the hair such as a 1.5 g hair tress. In another aspect, an effective amount of glucono-delta-lactone comprises applying about 0.2 grams to about 1.0 grams of at least about a 0.1 wt % aqueous glucono-delta-lactone solution to the hair such as a 1.5 g hair tress. In another aspect, an effective amount of glucono-delta-lactone comprises applying about 0.2 grams to about 2.0 grams of at least about a 2 wt % aqueous phytic acid solution to the hair such as a 1.5 g hair tress. In another aspect, an effective amount of glucono-delta-lactone comprises applying about 0.2 grams to about 2.0 grams of at least about a 1.5 wt % aqueous glucono-delta-lactone solution to the hair such as a 1.5 g hair tress. In another aspect, an effective amount of glucono-delta-lactone comprises applying about 0.2 grams to about 2.0 grams of at least about a 1 wt % aqueous glucono-delta-lactone solution to the hair such as a 1.5 g hair tress. In another aspect, an effective amount of glucono-delta-lactone comprises applying about 0.2 grams to about 2.0 grams of about 2 wt % to about 20 wt % aqueous glucono-delta-lactone solution to the hair such as a 1.5 g hair tress. In another aspect, an effective amount of glucono-delta-lactone comprises applying about 0.2 grams to about 1.5 grams of about 2 wt % to about 20 wt % aqueous glucono-delta-lactone solution to the hair such as a 1.5 g hair tress. In another aspect, an effective amount of glucono-delta-lactone comprises applying about 0.2 grams to about 1.0 grams of about 2 wt % to about 20 wt % aqueous glucono-delta-lactone solution to the hair such as a 1.5 g hair tress.

In another aspect, an effective amount of glucono-delta-lactone comprises applying about 0.2 grams to about 2.0 grams of about 2 wt % to about 15 wt % aqueous glucono-delta-lactone solution to the hair such as a 1.5 g hair tress. In another aspect, an effective amount of glucono-delta-lactone comprises applying about 0.2 grams to about 1.5 grams of about 2 wt % to about 15 wt % aqueous glucono-delta-lactone solution to the hair such as a 1.5 g hair tress. In another aspect, an effective amount of glucono-delta-lactone comprises applying about 0.2 grams to about 1.0 grams of about 2 wt % to about 15 wt % aqueous glucono-delta-lactone solution to the hair such as a 1.5 g hair tress. In another aspect, an effective amount of glucono-delta-lactone comprises applying about 0.2 grams to about 2.0 grams of about 2 wt % to about 10 wt % aqueous glucono-delta-lactone solution to the hair such as a 1.5 g hair tress. In another aspect, an effective amount of glucono-delta-lactone comprises applying about 0.2 grams to about 1.5 grams of about 2 wt % to about 10 wt % aqueous glucono-delta-lactone solution to the hair such as a 1.5 g hair tress. In another aspect, an effective amount of glucono-delta-lactone comprises applying about 0.2 grams to about 1.0 grams of about 2 wt % to about 10 wt % aqueous glucono-delta-lactone solution to the hair such as a 1.5 g hair tress. In another aspect, an effective amount of glucono-delta-lactone comprises applying about 0.2 grams to about 2.0 grams of about 2 wt %, about 5 wt %, about 10 wt %, about 15 wt %, or about 20 wt % aqueous glucono-delta-lactone solution to the hair such as a 1.5 g hair tress. In another aspect, an effective amount of glucono-delta-lactone comprises applying about 0.2 grams to about 1.5 grams of about 2 wt %, about 5 wt %, about 10 wt %, about 15 wt %, or about 20 wt % aqueous glucono-delta-lactone solution to the hair such as a 1.5 g hair tress. In another aspect, an effective amount of glucono-delta-lactone comprises applying about 0.2 grams to about 1.0 grams of about 2 wt %, about 5 wt %, about 10 wt %, about 15 wt %, or about 20 wt % aqueous glucono-delta-lactone solution to the hair such as a 1.5 g hair tress.

It will be appreciated that depending upon the pH of the medium, glucono-delta-lactone may exist as glucono-gamma-lactone or hydrolyzed forms such as gluconic acid and gluconate, each of which are encompassed by the term “glucono-delta-lactone.”

In one aspect, when used as component in a cosmetically acceptable composition, an effective amount of glucono-delta-lactone ranges from about 0.1 wt % to about 50 wt % based on the total weight of the cosmetically acceptable composition. In another aspect, when used as component in a cosmetically acceptable composition, an effective amount of glucono-delta-lactone ranges from about 0.1 wt % to about 20 wt % based on the total weight of the cosmetically acceptable composition. In another aspect, when used as component in a cosmetically acceptable composition, an effective amount of glucono-delta-lactone ranges from about 0.1 wt % to about 15 wt % based on the total weight of the cosmetically acceptable composition. In another aspect, when used as component in a cosmetically acceptable composition, an effective amount of glucono-delta-lactone ranges from about 2 wt % to about 50 wt % based on the total weight of the cosmetically acceptable composition. In another aspect, when used as component in a cosmetically acceptable composition, an effective amount of glucono-delta-lactone ranges from about 2 wt % to about 20 wt % based on the total weight of the cosmetically acceptable composition. In another aspect, when used as component in a cosmetically acceptable composition, an effective amount of glucono-delta-lactone ranges from about 2 wt % to about 15 wt % based on the total weight of the cosmetically acceptable composition. In another aspect, when used as component in a cosmetically acceptable composition, an effective amount of glucono-delta-lactone is about 0.5 wt %, about 1 wt %, about 2 wt %, about 5 wt %, or about 15 wt % based on the total weight of the cosmetically acceptable composition.

In one aspect, the pH of the solution comprising phytic acid or glucono-delta-lactone, or both, can be adjusted. For example, in certain aspects the pH of the solution comprising phytic acid or glucono-delta-lactone, or both, ranges from about pH 2 to about pH 10 e.g., from about pH 2 to about pH 5, from about pH 3 to about pH 5, and from about pH 4 to about pH 5. In certain aspects the pH of the solution comprising phytic acid or glucono-delta-lactone, or both, is about pH 2, about pH 3, about pH 4, about pH 4.5, or about pH 5.

Cosmetically acceptable compositions described herein may comprise, in addition to phytic acid or glucono-delta-lactone, or both, one or more cosmetically acceptable ingredients. Such ingredients include, but are not limited to, amino acids, amino acid derivatives, peptides, vitamins, keratins, acidifiers, polycarboxylic acids, fatty acids, fatty alcohols, fatty acid esters, emulsifiers, emollients, gelling agents, antioxidants, oils, waxes, preservatives, sunscreens, and polyphenols.

In one aspect, a cosmetically acceptable composition may comprise the following and phytic acid in the appropriate amount.

| | | preferred |
|---|---|---|
| Ingredients | % w/w range | % w/w range |
| Water | q.s. | q.s. |
| Esters | 1.00-10.00 | 3.50-6.00 |
| Fatty Alcohol | 1.00-8.00 | 2.00-4.50 |
| Organic Salt | 0.10-5.00 | 0.50-3.50 |
| Alcohol | 0.10-5.00 | 1.00-2.50 |
| Alkoxylated Alcohol | 0.10-5.00 | 0.25-2.00 |
| Amino Acid | 0.10-5.00 | 0.25-2.00 |
| Gum | 0.01-3.50 | 0.50-2.00 |
| Polyol | 0.01-2.00 | 0.20-1.50 |
| Botanical Derivative | 0.01-2.00 | 0.25-1.00 |
| Fragrance | 0.01-1.00 | 0.20-0.75 |
| Carboxylic Acid | 0.01-1.00 | 0.10-0.60 |
| Phenol | 0.01-1.00 | 0.10-0.60 |
| Oil | 0.01-1.00 | 0.01-0.60 |
| Inorganic Base | 0.01-1.00 | 0.01-0.60 |
| Carbohydrate | 0.01-1.00 | 0.01-0.60 |

In the foregoing table it is to be understood that the % w/w ranges includes all ranges and individual % w/w amounts in between. For example, 0.10% to 0.60% wt/wt includes 0.10% to 0.50% wt/wt, . . . 0.10% to 0.40% wt/wt, . . . 0.10% to 0.30% wt/wt, . . . 0.10% to 0.20% wt/wt . . . , etc., and 0.10% wt/wt, . . . 0.20% wt/wt, . . . 0.30% wt/wt, . . . 0.40% wt/wt, . . . 0.50% wt/wt, . . . 0.60% wt/wt, etc.

As discussed above, and shown in the exemplification section below, applying phytic acid or glucono-delta-lactone, or both, to the hair results in improvements in the thermal properties of the hair e.g., an increase in denaturation temperature and/or better humidity resistance. In some aspects, the increase in denaturation achieved following the application of phytic acid or glucono-delta-lactone, or both to the hair is 3° C. or more, 4° C. or more, or 5° C. or more.

Also, as discussed above, and shown in the exemplification section below, applying phytic acid or glucono-delta-lactone, or both, to the hair results in improvements in the mechanical properties of the hair e.g., an increase in break stress and/or Young's modulus. In some aspects, the increase in break stress achieved following the application of phytic acid or glucono-delta-lactone, or both to the hair is 3% or greater, 4% or greater, 5% or greater, 6% or greater, 7% or greater, 8% or greater, or 9% or greater. In some aspects, the increase in break stress achieved following the application of phytic acid or glucono-delta-lactone, or both to the hair ranges from 3% to about 30% or from about 5% to about 30%. In some aspects, the increase in Young's modulus achieved following the application of phytic acid or glucono-delta-lactone, or both to the hair is 3% or greater, 4% or greater, 5% or greater, 6% or greater, 7% or greater, 8% or greater, or 9% or greater. In some aspects, the increase in Young's modulus achieved following the application of phytic acid or glucono-delta-lactone, or both to the hair ranges from 3% to about 30% or from about 5% to about 30%.

Also, as discussed above, and shown in the exemplification section below, applying phytic acid or glucono-delta-lactone, or both, to the hair results in an increase in the surface hydrophobicity of the hair. In some aspects, the increase in surface hydrophobicity achieved following the application of phytic acid or glucono-delta-lactone, or both to the hair is defined by an increase in the water contact angle as measured by differential wetting characterization (DWC).

Also, as discussed above, and shown in the exemplification section below, applying phytic acid or glucono-delta-lactone, or both, to the hair minimizes protein loss. In certain aspects, this minimization is defined as a greater than 25% reduction in the protein loss as measured by Modified Lowry Protein Assay.

### EXEMPLIFICATION

The following examples are provided by way of illustration and not limitation.

### Treatment of Damaged Hair with Phytic Acid (Pa) and Phytic Acid-Gluconolactone (Pa-Gl) Combination

### Treatment on Bleached Hair

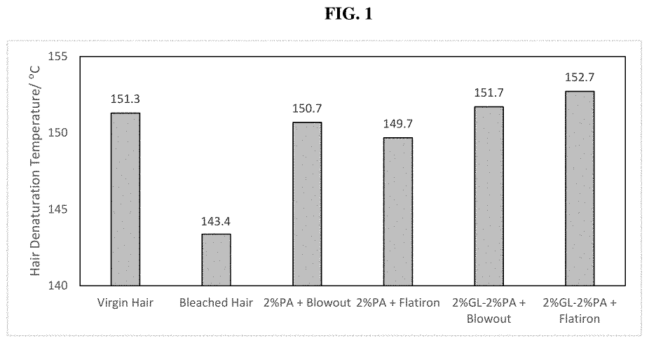

To explore treatment with PA and PA-GL, studies were performed using 3× bleached hair as a damaged hair model (each bleaching was done with a mixture of Bleaching powder (BW2) and 40V clear developer at 1:2 ratio, for 1 hour at room temperature). The bleached hair tresses were treated with a 2 wt % PA aqueous solution at pH 2, for 15 min followed by heat styling (blow dry or flat iron at 450° F.). Differential scanning calorimetry (DSC) was performed on tresses at wet stage to determine the effect of phytic acid treatment on hair denaturation temperatures (Td). For wet stage DSC, a hair sample of 3-5 mg in 50 μL Deionized water sealed and equilibrated (overnight) in a stainless steel pressure resistant high-volume pan is heated from 25° C. to 180° C. at a 5 ° C./min heating rate. FIG. 1 shows denaturation temperatures of the untreated virgin hair, 3× bleached and 3× bleached hair treated with 2 wt % PA aqueous solution. The bleached hair showed a much lower Td than the virgin hair, suggesting severe damage caused by the bleaching process. However, once the bleached hair was further treated with 2 wt % PA aqueous solution, the hair denaturation temperature fully recovered to the virgin level, suggesting the strong internal structural repair and strengthening benefits of PA. The Td remained similar (≥149° C.) even after 10 shampoo and conditioner washing cycles, confirming the strong interaction between PA and hair fibers demonstrating the long-lasting strengthening benefits provided by the treatment.

To further boost the strengthening benefits, the use of PA together with glucono-delta-lactone, which has the potential to cross link with PA and hair functional groups, especially under high heat conditions was also explored. In one example, hair was treated with an aqueous solution comprising 2 wt % PA and 2 wt % GL, at pH 2 for 15 min followed by blow drying or flat ironing. As seen in FIG. 1, once the bleached hair was treated with PA-GL, the hair denaturation temperature attained a level beyond that of virgin hair, suggesting a strong repair and strengthening power by the PA-GL blend. Very similar to the phytic acid system, the improved Td by PA-GL remained similar for at least 10 shampoo and conditioner washing cycles, demonstrating the strong interaction between PA-GL and hair fibres.

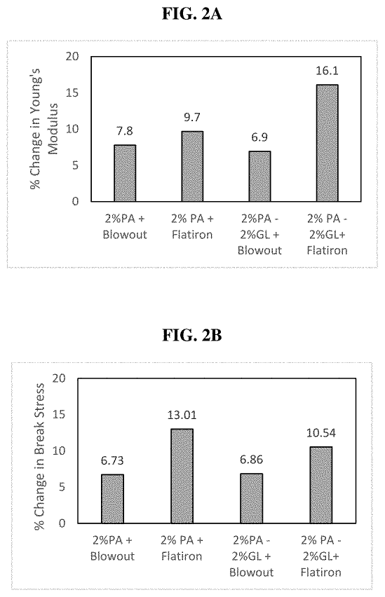

Additionally, the strengthening benefits of PA and PA-GL blend treatments were confirmed by Dia-Stron single fiber mechanical testing at wet stage. For wet stage single fiber method, hair fibers (≥20 fibers per treatment) at 30 mm length are fixed by 2 brass crimps at the ends and mounted on a Dia-stron tray. Initial dimension measurements are collected using a Dia-Stron FDAS770 (double ended samples, sample size of 30 mm, no of slices of 5 under one rotation per slice), followed by equilibration in Deionized water for at least 2 h prior to testing. All fiber tensile measurements are performed using a Dia-Stron MTT686/MTT690 (under 100% extension at gauge force of 2N, break threshold of 5 g, rate of 20 mm/min, maximum force of 2000 gmf and premature failure threshold of 10% strain). FIGS. 2A and 2B show that both Young's modulus and break stress were greatly improved when bleached hair was treated with PA or PA-GL followed by heat styling (blow dry or flatiron). The maximal strengthening was achieved for PA-GL with flat ironing, suggesting the effective cross-linking between PA and GL and with hair functional groups at an elevated temperature.

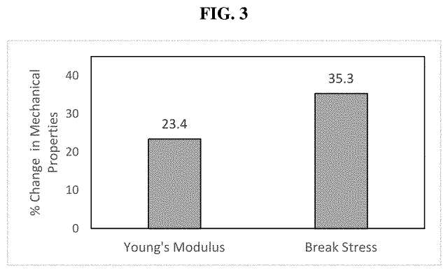

In another example, a more damaged hair model was prepared by two times bleaching treatments under heat, each at 45° C. for 1 hour with a mixture of Bleaching powder (BW2) and 40V clear developer at 1:2 ratio. FIG. 3 shows that both Young's modulus and break stress were greatly improved after a treatment with 2 wt % PA aqueous solution at pH 2 for 1 hour.

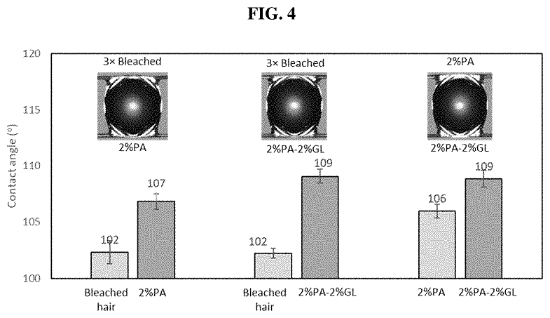

To demonstrate the surface repairing capabilities of PA and PA-GL systems, a differential wetting characterization (DWC) technique was used to evaluate hair surface hydrophobicity change before and after treatments. In this method, a pair of hair fibers is mounted on an in-house built stage in a horizontal plane with a separation of ˜0.75 mm. A droplet of deionized water (1 μL) is then applied onto the fiber pair by using a micro syringe. The droplet is observed using a Nikon microscope under 5× magnification and the image is taken using the incorporated digital camera. In the image, the contact angles of the water droplet on the fiber pair are measured respectively using the Nikon Advance Research software. The relative surface hydrophobicity of the fiber pair is correlated to the resulting contact angles, of which the greater value indicates higher hydrophobicity. The water contact angles were measured on 3× bleached hair treated with 2 wt % PA aqueous solution and 2% wt PA-GL aqueous solution with respect to the untreated bleached hair. A low water contact angle was observed for the bleached hair, suggesting that bleached hair surface was relatively hydrophilic. As shown in FIG. 4, a 5° increase in water contact angle was achieved upon treatment with a 2 wt % PA aqueous solution, suggesting a significant improvement in hair surface hydrophobicity. The hydrophobicity is further improved (7° increase) by using a PA-GL blend solution. The higher water contact angle of the PA-GL treated fiber with respect to the PA treated fiber suggests additional benefits by the incorporation of GL.

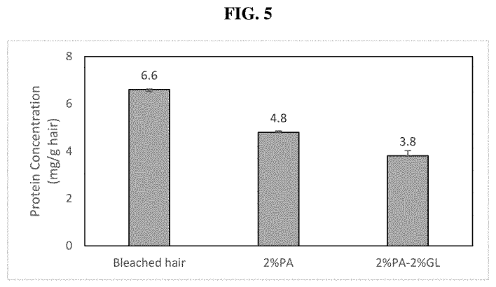

In addition, surface repair benefits from the PA or PA-GL treatments were evaluated by a protein loss assay. It is known that hair cuticle damage after various chemical treatments such as bleaching and perming results in a higher protein loss. To quantify the protein loss before and after treatments, a Modified Lowry Protein Assay was employed. In this method, a Lowry assay (Pierce Modified Lowry Protein Assay Kit, working range is at 1-1500 μg/mL) followed by absorbance measurement using UV-Visible Spectrophotometer (λmax at 740-760 nm) is used to quantify hair protein loss for a sample size of 250 mg of hair in 4 mL of deionized water. As shown in FIG. 5, bleached hair is susceptible to protein loss, demonstrated by a high protein loss of 6.6 mg/g hair. However, once the bleached hair was treated with PA or PA-GL, the protein loss was significantly reduced by 27% and 42%, respectively.

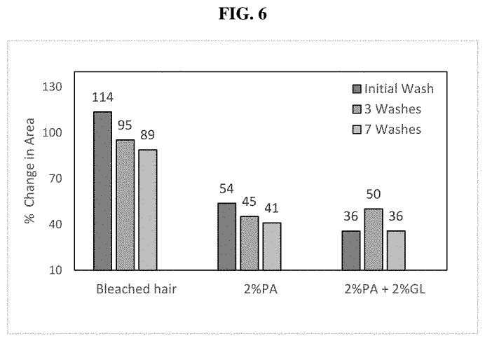

Both the internal structural strengthening and the surface repairing provided by PA and PA-GL systems translated to a superior humidity resistance benefit. In this method, hair tresses are exposed to high humidity at 65% RH at 25° C. for 30 min and the area of hair tresses before and after exposure to humidity is quantified using Image J application. As shown in FIG. 6, when exposed to a high humidity test, hair tresses treated with both systems exhibited much less area increase compared to the untreated bleached hair, suggesting superior frizz protection benefit provided by PA and PA-GL blend. The differences remained significant even after 7 shampoo and conditioner washing cycles, suggesting long-lasting humidity resistance benefits provided by both PA based systems.

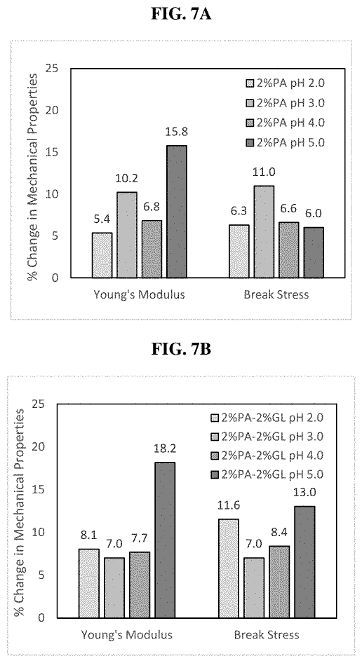

To better understand the impact of pH on the hair strengthening benefits, treatments with pH ranging from 2 to 5 were also investigated. FIGS. 7A and 7B show that, under all conditions, both Young's modulus and break stress were greatly improved when bleached hair was treated with PA or PA-GL followed by heat styling (flatiron), suggesting both systems promote effective cross-linking with hair functional groups over the entire pH range explored.

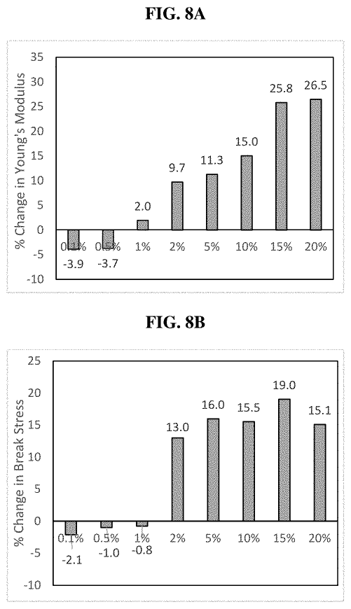

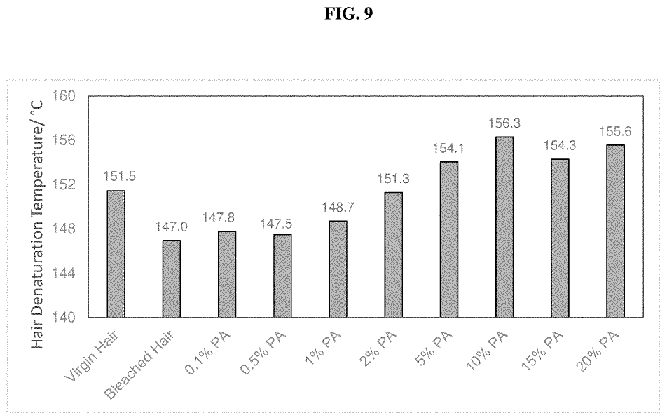

Treatments with PA concentrations varying from 0.1 to 20 wt % were also explored. In all tests, 3× bleached hair tresses were treated with PA aqueous solutions for 15 min followed by heat styling. For the high concentration (i.e., 10, 15, 20 wt %) treatments, a quick 20 s water rinse was also added before heat styling. FIGS. 8A and 8B show hair mechanical properties change upon PA treatments. It is clear that significant improvement in Young's modulus and break stress was only achieved when the PA concentration reached 2 wt % or above. A clear positive dose response relationship between PA concentration and hair denaturation temperature was also observed. Similarly, FIG. 9 shows that the denaturation temperature of hair treated with PA at 2 wt % or a higher concentration recovered back to or even beyond the virgin level suggesting significant strengthening benefits achieved.

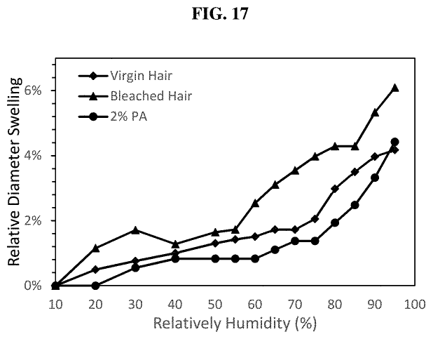

To further demonstrate repair benefits from PA treatment, relative hair diameter swelling rates were measured at various relative humidity (RH) levels using environmental scanning electron microscopy. All measurements were carried out using a FEI Quanta 200 FEG MKII scanning electron microscope, operating at 15 kV, with relative humidity ranging from 10% to 95%. Single source virgin brown hair was bleached three times to obtain a 3× bleached hair model (each bleaching was done with a mixture of bleaching powder (BW2) and 40V clear developer at 1:2 ratio for 1 hour at room temperature, followed by 30 s water rinse and air-dry at room temperature). Compared to virgin hair, the 3× bleached hair showed a greater increase in diameter swelling with increasing relative humidity from 10% to 95% RH (FIG. 17) due to higher water absorption rate, indicating increased porosity and damage for the bleached hair. However, once the bleached hair was treated with a 2 wt % PA aqueous solution at pH 4-4.5 for 30 min followed by blow dry, the relative diameter swelling rate as greatly reduced, even lower than virgin hair.

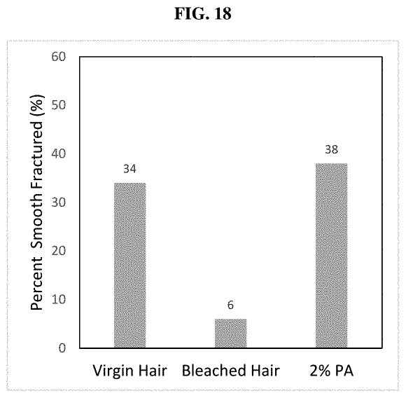

Depending on the level of hair damage, hair fibers may break with very different fracture patterns when pulling to break. For example, Caucasian hair shows smooth fracture as the predominant mode of fracture, whereas more damaged textured hair tends to break with non-uniform fracture patterns like step fractures. Fracture patterns of fibers broken from Dia-stron tensile testing were examined under optical microscope to understand the relationship between fracture pattern and hair damage level. As shown in FIG. 18, virgin hair breaks with approximately 34% smooth fractures. However, the 3× bleached hair only shows 6% smooth fractures. In contrast, when the bleached hair was treated with a 2 wt % PA aqueous solution at pH 4-4.5 for 10 min, followed by blow dry, 38% smooth fractures were observed, close to that for virgin hair, indicating effective internal hair structure repair and complete restore by PA.

### Treatment on Reduced Hair

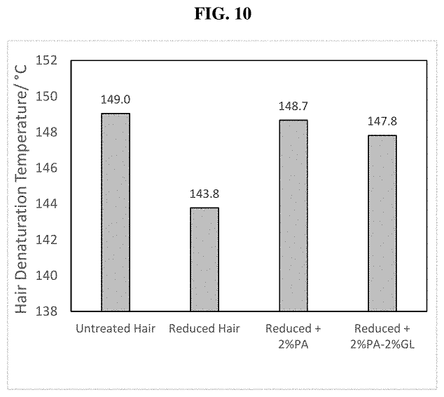

The use of PA and PA-GL as a stand-alone treatment on reduced hair was also investigated. For hair reduction, a hair tress (1.5 g) was treated with a solution of 5% ammonium thioglycolate (ATG) at pH 9.5, massaged thoroughly (˜1 min) onto the hair tress and kept for 20 min. After ATG treatment, hair tress was rinsed with water for 30 s, towel dried and post-treated with 0.5 g of 2 wt % PA aqueous solution at pH 2, for 5 min followed by blow drying. FIG. 10 shows hair denaturation temperatures of untreated virgin hair, ATG-reduced and ATG-reduced hair treated with 2 wt % PA. The denaturation temperature significantly decreased after hair reduction, signifying significant damage caused by ATG. However, once the reduced hair was treated with PA or PA-GL post-treatment the hair denaturation temperature recovered to the level of virgin hair, demonstrating the restoring and strengthening benefits by PA and PA-GL.

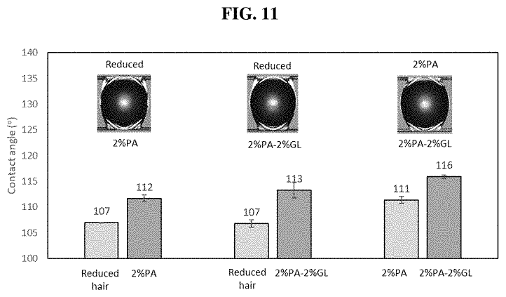

The surface repair capabilities by PA and PA-GL on reduced hair were also evaluated using DWC measurements. As shown in FIG. 11, a 5° increase in water contact angle is achieved after treatment with 2 wt % PA solution post reduction, indicating an improvement in hair hydrophobicity. The hydrophobicity is further enhanced (6° increase in water contact angle) by using PA-GL blend solution.

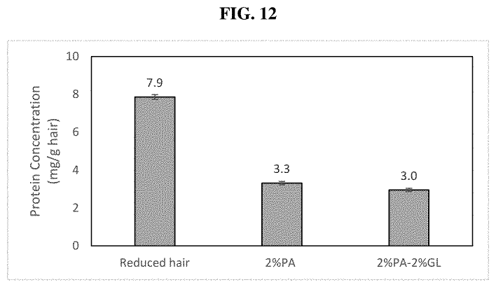

As shown in FIG. 12, hair was treated with ATG became highly susceptible to protein loss, as indicated by a high protein loss of 7.9 mg/g hair. When the reduced hair is treated with PA and PA-GL systems, the protein loss was significantly reduced by 58% and 62%, respectively.

### Treatment on Heat Damaged Hair and Protection Benefits

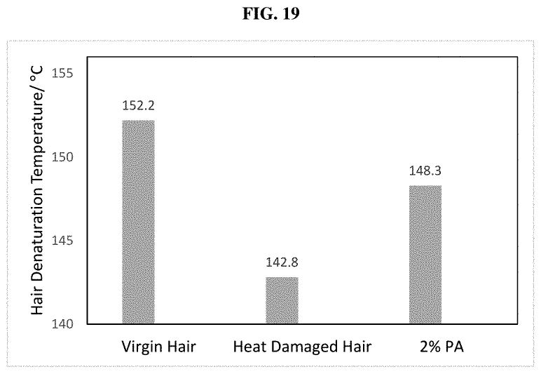

To demonstrate the repair benefits PA provides for heat damaged hair, hair denaturation temperatures (Td) of virgin hair, heat damaged hair, and heat damaged and PA treated hair were measured using Differential scanning calorimeter (DSC). The heat damaged hair was prepared by subjecting virgin brown hair to 4 cycles of heat treatments. In each cycle, hair was treated with Deionized water (1 mL per 1.5 g of hair), massaged for 30 s followed by heat treatment (blow dry until dry at medium heat setting, flat iron at 450° F. for 10 strokes at a rate of 10 seconds per stroke from root to tip). At the end of 4 cycles of heat treatments, the hair tress is dialyzed overnight in Deionized water and air dried before DSC testing. The PA treatment on the heat damaged hair was performed using a 2 wt % PA aqueous solution at pH 4.5 for 30 min followed by blow dry. As shown in FIG. 19, 4 cycles of heat treatment resulted in a 9° C. decrease in Td. However, upon the PA treatment, 5° C. recovery in Td was achieved, suggesting the effective heat damage repair by PA.

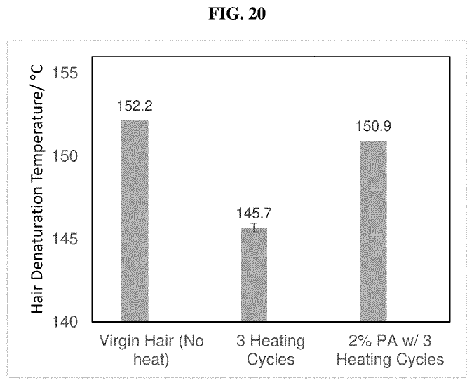

The heat protection benefit provided by PA using DSC was also explored, for which virgin hair with or without PA pre-treatment was subjected to 3 cycles of heat treatments. A virgin control sample was also added for comparison, which was prepared by treating the virgin brown hair with Deionized water (1 mL per 1.5 g of hair), massaged 30 seconds and air dried. For each heat treatment cycle, hair sample was blow dried until dry at medium heat setting, and flat ironed at 450° F. for 10 strokes at a rate of 10 seconds per stroke from root to tip. After three cycles of heat treatments, hair samples were dialyzed overnight in DI water and air dried before DSC testing. The DSC analysis (FIG. 20) shows that 3 cycles of heat treatments led to a 6.5° C. decrease in Td compared to the virgin hair. However, the PA pretreated hair didn't show any significant change in Td upon 3 cycles of heat treatments, indicating the effective heat protection benefit provided by PA.

### Phytic Acid (Pa) and Phytic Acid-Gluconolactone (Pa-Gl) as a Post-Treatment for Color Service

Phytic acid also provides benefits as a post-treatment for salon color treatments. In this study, the color treatment was performed on a 3× bleached hair with a Wella permeant hair color product (Ion Color Brilliance permanent crème hair color, 7RR, Intense Red), which consists of hair colorants and Wella professional's (Welloxon Perfect™) developer (H2O2, 20 Vol-6%). The final color mixture was mixed for 1 min., massaged thoroughly (˜1 min) onto 3× bleached hair tress (1.5 g) and kept for 30 min. color development. After color development, the hair tress was rinsed with water for 30 s, towel dried and post-treated with 0.5 g of 2 wt % PA aqueous solution at pH 2 for 5 min. followed by blow dry styling.

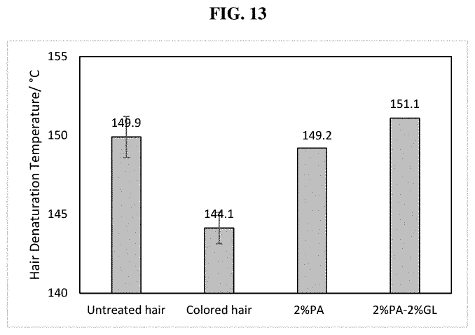

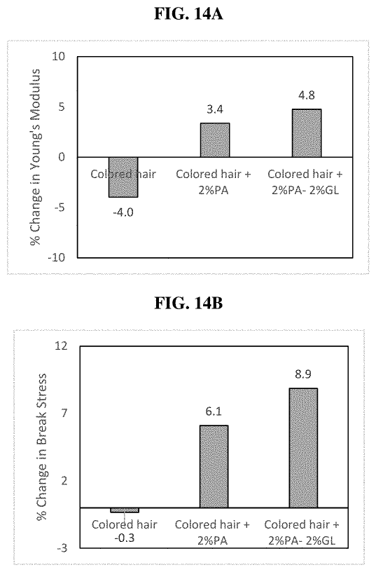

FIG. 13 shows hair denaturation temperatures of hair tresses treated with color followed by 2 wt % PA as compared to untreated and colored hair. The colored hair exhibited a much lower Td than the untreated virgin hair. However, once the colored hair is post-treated with PA the hair denaturation temperature recovers to the level of the virgin hair, suggesting the repairing and strengthening benefits by PA. Similarly, Dia-stron single fiber mechanical testing shows that both Young's modulus and break stress were greatly improved after the post-treatment with PA (FIG. 14A and FIG. 14B). Additionally, as shown in FIGS. 13, 14A, and 14B, both Td and mechanical properties are further improved by incorporating GL with PA.

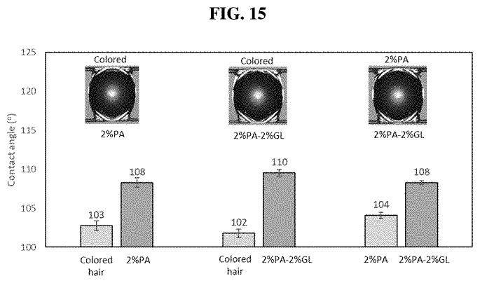

In another study, the water contact angles were measured on colored hair fibers before and after the treatment with 2 wt % PA aqueous solution or 2 wt % PA-GL aqueous solution. As shown in FIG. 15, a 5° increase in water contact angle was achieved upon the treatment with 2 wt % PA solution, suggesting a significant improvement in hair surface hydrophobicity. The hydrophobicity is increased further (8° increase in water contact angle) by using the PA-GL blend solution. Together with DSC and Dia-Stron data, these results suggest PA and PA-GL as effective post-treatments for color services, providing clear internal structural strengthening and surface repair benefits.

### Delivery and Testing of Phytic Acid (Pa) as Hair Care Formulation

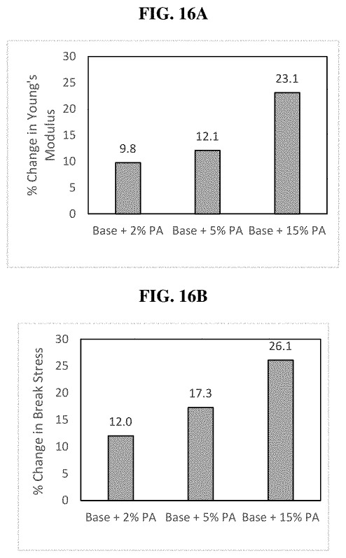

The initial delivery of PA in hair care formulations was studied. Phytic acid was incorporated into Restore Repair Leave-In conditioner by Living Proof at a 2 wt %, 5 wt %, or 15 wt % concentration based on the total weight of the components in the conditioner. As shown in FIG. 16A and FIG. 16B, at all three PA concentrations, significant increases in both Young's modulus and break stress were achieved with a positive dose response relationship, suggesting efficient delivery of PA and thus effective repair and strengthening in the leave-in conditioner.

### A. Improved Hair Strength & Structural Integrity

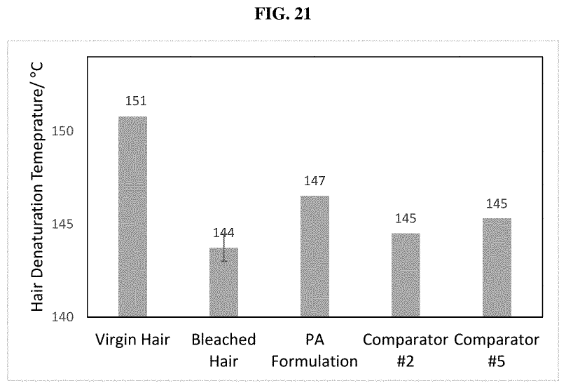

To explore strengthening benefits of PA in a hair care formulation, a PA formulation containing 0.75 wt % PA at pH 4-4.5 was used as an example. The 3× bleached hair was treated with 0.25 g of the PA formulation for 10 min followed by blow dry. Differential scanning calorimetry was performed on tresses to determine the effect of phytic acid treatment on hair denaturation temperatures (Td). As shown in FIG. 21, the bleached hair showed a much lower Td than the virgin hair, suggesting severe damage caused by the bleaching process. However, once the bleached hair was further treated with the PA formulation, the hair denaturation temperature increased by 3° C., suggesting the significant internal structural repair and strengthening benefits of PA when delivered in a hair care formulation. Additionally, the repair benefit is superior to two bond builder comparator formulations (#2 & #5) as shown in FIG. 21.

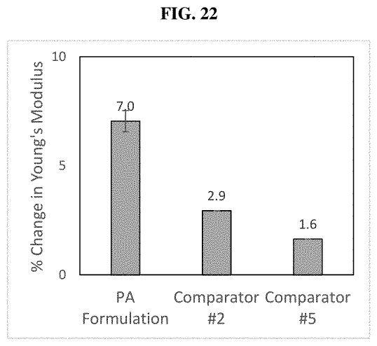

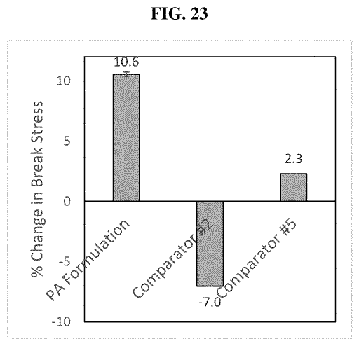

The strengthening benefit of the PA formulation treatment was further confirmed by Dia-Stron single fiber mechanical testing. FIGS. 22 and 23 show that both Young's modulus and break stress were greatly improved when 3× bleached hair was treated with the PA formulation followed by blow dry. In contrast, the comparator formulations showed little or no improvements. These results together with the DSC data suggest efficient delivery of PA and thus effective repair and strengthening benefits in the hair strengthening formulation.

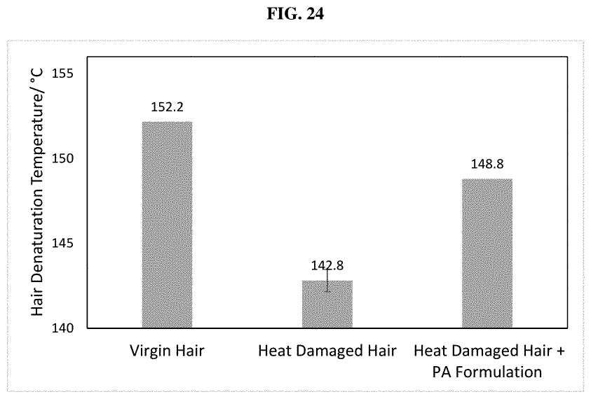

To demonstrate how PA formulation repairs heat damaged hair, the hair denaturation temperature (Td) of virgin hair, heat damaged hair, and heat damaged hair treated with PA formulation (0.75 wt % PA, 0.25 g) at pH 4-4.5 for 10 min, followed by blow dry was compared using Differential scanning calorimeter (DSC). The heat damaged hair was prepared by subjecting virgin brown hair to 4 cycles of heat treatments. In each cycle, hair was treated with Deionized water (1 mL per 1.5 g of hair), massaged for 30 s followed by heat treatment (blow dry until dry at medium heat setting, flat iron at 450° F. for 10 strokes at a rate of 10 seconds per stroke from root to tip). At the end of 4 cycles of the heat treatments, the hair tress is dialyzed overnight in Deionized water and air dried before DSC testing. As shown in FIG. 24, 4 cycles of heat damage resulted a 9° C. decrease in Td. However, PA treatment on the heat damaged hair resulted in a significant recovery of hair Td (6° C. increase), suggesting heat damage repair benefit of PA in the formulation form.

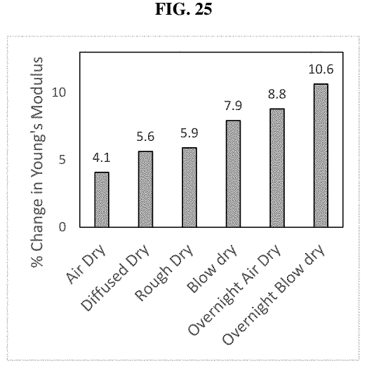

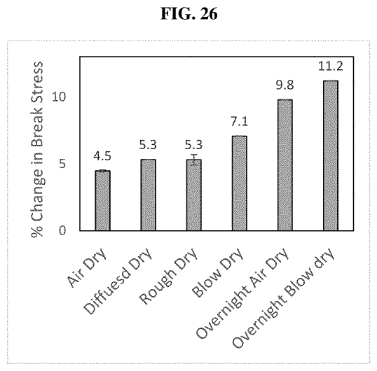

To demonstrate the versatile use of PA formulation, Dia-Stron single fiber mechanical testing was carried out on 3× bleached hair treated with the PA formulation followed by different styling methods (air dry, diffuse dry, rough dry, blow dry, overnight air dry and overnight blow dry). As shown in FIGS. 25 and 26, significant improvement in both Young's modulus and break stress were achieved by all different styling methods and suggesting the versatile use of the PA formulation for hair repair and strengthening.

To further demonstrate cortex to cuticle strengthening benefit of PA formulation, hair life cycle rig (HLCR) test was performed on Virgin European Brown hair (5 g, 10″ wide metal clamp), 1× bleached hair, and 1× bleached hair treated with PA formulation or comparator products. The 1× bleached hair was prepared by submerging 1.5 pounds of hair in a 8400 mL of bleaching solution consisting of 14% Ammonia (6% concentrate), 29% Peroxide (34% concentrate) and 57% cold water for 1 h and 45 mins, washed with Texapon ES2 and water, and dried at room temperature. The HLCR test was carried out at a rate of 60 combs per min for a total of 10,000 combing cycles. The number of broken fibers was counted after every 200 combing cycles. Ten tresses were included for each treatment condition. Both virgin and 1× bleached hair were treated with basic shampoo and conditioner (each 0.75 g per 5 g tress) only. The PA formulation treated hair sample was prepared by treating 1× bleached hair with basic shampoo and conditioner, followed by 0.83 g of formulation for 10 min and blow dry. Similarly, comparator #2 sample was prepared by treating hair with basic shampoo followed by 0.83 g of product formulation for 4 min and blow dry; and comparator #5 sample was prepared by treating hair with 3.33 g of product formulation for 10 min followed by basic shampoo, conditioner wash and blow dry.

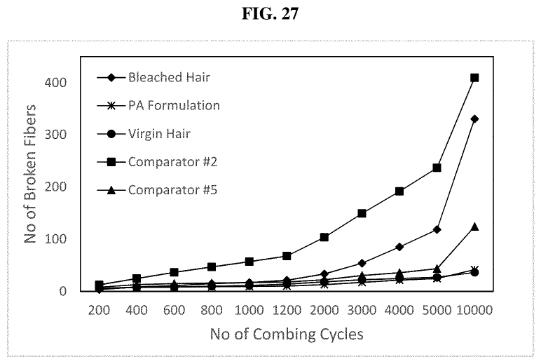

As shown in the FIG. 27, compared to the bleached and the two comparators, the PA formulation treated hair shows the least number of broken hair fibers at different combing cycle intervals up to 10,000 combing cycles, suggesting the effective strengthening by PA and superior performance compared to the comparator formulations. It is worth noting that the PA formulation treated hair performed almost identical to the virgin hair in this test, suggesting that the PA formulation was able to restore hair back to the virgin state. Further quantification shows that the PA formulation makes hair 8× more resistant to breakage, resulting in an 87% less fiber breakage. In other words, the hair becomes 8× stronger after the PA formulation treatment.

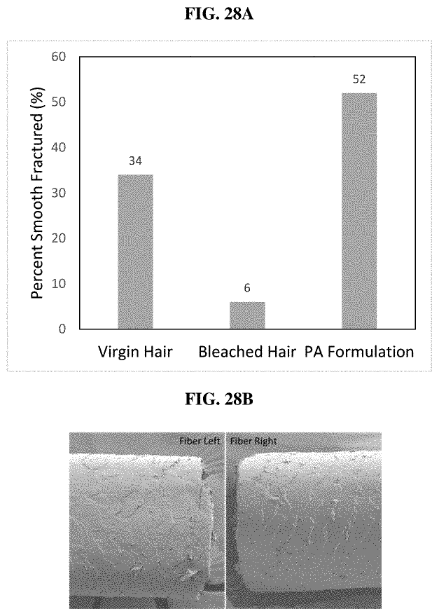

Hair fracture patterns of 3× bleached and PA formulation treated fibers after Dia-stron tensile testing were also examined and compared to those for virgin and bleached hair. As shown in FIG. 28A, PA treatment of bleached hair leads to a much higher percentage of smooth fracture patterns, even more than virgin hair, suggesting the improved internal hair structure after the treatment. FIG. 28b shows representative SEM images of smooth fracture patterns obtained in the PA formulation treated hair.

### B. Cuticle & Surface Repair

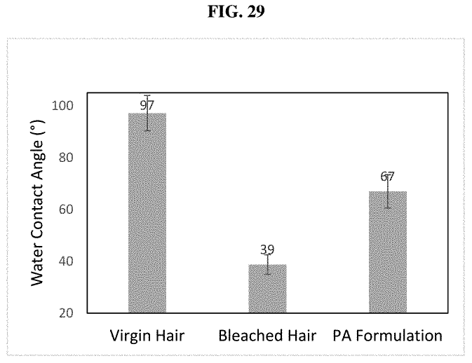

To demonstrate the surface repairing capabilities of PA formulation, Kruss DSA-100 M drop shape analyzer was used to evaluate the absolute hair surface hydrophobicity change before and after treatments by measuring water contact angles. In this method, a droplet of Deionized water (20-60 pl) is applied onto the hair fiber using the Microdrop digital dosing device and the evolution of the droplet on the hair is automatically recorded in the Kruss Advance software. The surface hydrophobicity of the hair fiber is determined by the initial contact angle of the water droplet on the hair fiber. The greater value of the contact angle indicates higher hydrophobicity. A low water contact angle was observed for the 3× bleached hair and the 3 cycles of bleaching reduced surface hydrophobicity by about 60% compared to virgin hair (FIG. 29). However, the contact angle increased by about 50% after the PA formulation treatment, indicating a significant improvement in hair surface hydrophobicity.

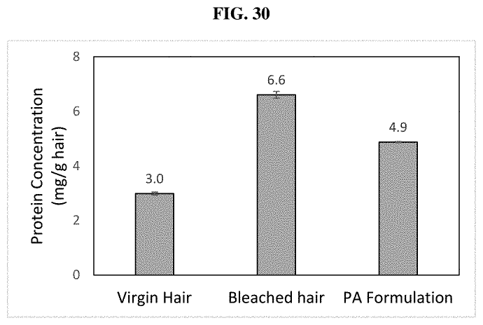

In addition, surface & cuticle repair benefits from PA formulation were evaluated by a protein loss assay. It is known that a higher protein loss is resulted from hair cuticle damage after various chemical treatments like bleaching. To quantify the protein loss before and after treatments, a Modified Lowry Protein Assay was employed. As shown in FIG. 30, 3× bleached hair is prone to protein loss, demonstrated by a high protein loss of 6.6 mg/g hair. However, once the bleached hair was treated with PA formulation, the protein loss was significantly reduced to 4.9 mg/g hair.

### C. Protection Against Future Damage

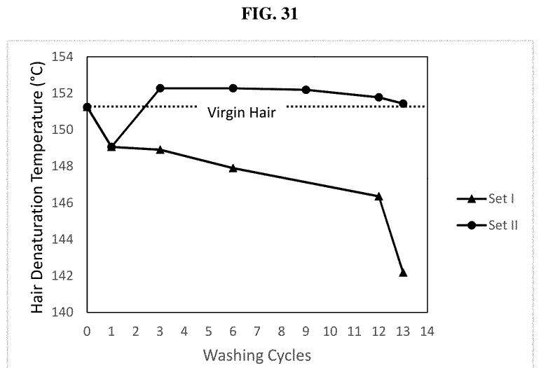

Furthermore, the capability of PA formulation to protect hair from future chemical treatments (bleaching and coloring) was explored. In this study, two sets of treatments were performed and for each set three replicates were included. In Set I, a virgin tress was bleached once (1^{st} bleach), followed by 12 cycles of shampoo and conditioner washes, then another bleach (2^{nd} bleach). In Set II, similar bleaching and washing process was taken except that the tress was treated with PA formulation after the 1^{st} bleach and 3^{rd}, 6^{th}, 9^{th}, and 12^{th} wash. In both bleaching steps, hair was treated with a mixture of BW2 bleaching powder and clear 30 V developer (1:1.5 ratio, 5 g per 1.5 g of hair) for 20 min under heat (high heat setting in a Belvedere hooded dryer), followed by 30 s water rinse and air dry at room temperature. The hair denaturation temperature (Td) was collected after each bleach treatment and every 3 washing cycles. As shown in FIG. 31, in Set I, the Td decreased by 3° C. after 1^{st} bleach and continued to decrease over 12 washing cycles and after 2^{nd} bleach (9° C. decrease in total). On the other hand, in Set II, the hair tress treated with PA formulation after 1^{st} bleach showed immediate improvement in Td (3° C.), even beyond the virgin state, and remained above the virgin level over 12 washing cycles and even after 2^{nd} bleach. These results clearly demonstrate the instant repair by PA as well as effective protection from chemical treatments related future damage.

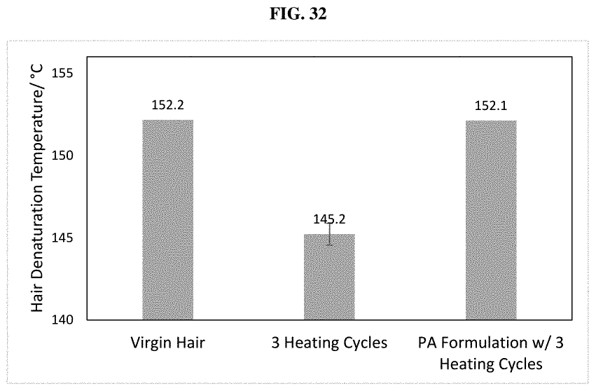

Heat protection provided by the PA formulation using DSC was also explored. Here, three groups of hair samples were compared: virgin, heat control, and PA formulation treated. The virgin hair was treated with DI water (1 mL per 1.5 g of hair), massaged 30 s and air dried. The heat control sample was prepared by treating a virgin brown tress with DI water (1 mL per 1.5 g of hair), massaged 30 s followed by one cycle of heat treatment (blow dry until dry at medium heat setting, flat iron at 450° F. for 10 strokes at a rate of 10 seconds per stroke from root to tip). The PA formulation treated sample was prepared by treating a virgin brown tress with PA formulation (0.25 g per 1.5 g of hair), massaged 30 s followed by the same heat treatment. Each hair sample was subjected to 3 cycles of treatments, followed by overnight dialysis in DI water, and air dried before DSC testing. The results (FIG. 32) show significant differences among the 3 groups of samples. Virgin hair has the highest denaturation temperature. The heat control (3 heating cycles) shows a 7° C. decrease in Td. However, the PA formulation treated hair has the same Td as virgin hair even after 3 heating cycles, indicating future protection from heat damage by PA treatment in formulation form.

1. A method of strengthening or repairing hair comprising applying to the hair an effective amount of phytic acid.

2. (canceled)

3. The method of claim 1, further comprising applying to the hair an effective amount of phytic acid and an effective amount of glucono-delta-lactone.

4. The method of claim 1, wherein the hair is human hair.

5. The method of claim 1, wherein the hair has been bleached prior to applying the phytic acid or the combination of phytic acid and glucono-delta-lactone.

6. The method of claim 1, wherein the hair has been reduced prior to applying the phytic acid or the combination of phytic acid and glucono-delta-lactone.

7. The method of claim 1, wherein the hair has been color treated prior to applying the phytic acid or the combination of phytic acid and glucono-delta-lactone.

8. The method of claim 1, further comprising the step of heating the hair after applying phytic acid or the combination of phytic acid and glucono-delta-lactone.

9. The method of claim 1, wherein the phytic acid is present as part of an aqueous solution.

10. The method of claim 1, wherein the effective amount of phytic acid comprises applying at least about 0.5 wt % aqueous phytic acid solution to the hair.

11. The method of claim 1, wherein the effective amount of phytic acid comprises applying at least about 2 wt % aqueous phytic acid solution to the hair.

12. The method of claim 1, wherein the effective amount of phytic acid comprises applying about 0.5 wt % to about 50 wt %, about 2 wt % to about 50 wt %, about 2 wt % to about 20 wt %, about 2 wt % to about 15 wt %, about 2 wt % to about 10 wt %, about 0.5 wt % to about 1 wt %, about 1 wt % to about 3 wt %, about 0.75 wt %, about 2 wt %, about 5 wt %, about 10 wt %, about 15 wt %, or about 20 wt % aqueous phytic acid solution to the hair.

13. The method of claim 1, wherein the effective amount of phytic acid comprises applying about 2 wt % aqueous phytic acid solution to the hair.

14. The method of claim 1, wherein the effective amount of phytic acid comprises applying about 0.75 wt % aqueous phytic acid solution to the hair.

15. The method of claim 3, wherein the effective amount of glucono-delta-lactone comprises applying at least about 2 wt % aqueous glucono-delta-lactone solution to the hair.

16. The method of claim 3, wherein the effective amount of glucono-delta-lactone comprises applying about 0.5 wt % to about 50 wt %, about 0.5 wt % to about 20 wt %, about 0.5 wt % to about 15 wt %, about 0.5 wt % to about 10 wt %, about 0.5 wt %, about 1 wt %, about 2 wt %, about 5 wt %, about 10 wt %, about 15 wt %, or about 20 wt % aqueous glucono-delta-lactone solution to the hair.

17. The method of claim 3, wherein the effective amount of glucono-delta-lactone comprises applying a 2 wt % aqueous glucono-delta-lactone solution to the hair.

18. The method of claim 3, wherein the glucono-delta-lactone is applied concurrently with the phytic acid.

19. The method of claim 9, wherein the pH of the solution ranges from about pH 2 to about pH 10, from about pH 2 to about pH 5, from about pH 3 to about pH 5, or from about pH 4 to about pH 5.

20. The method of claim 9, wherein the pH of the solution is about pH 2, about pH 3, about pH 4, about pH 4.5 or about pH 5.

21. The method of claim 1, wherein the phytic acid or the combination of phytic acid and glucono-delta-lactone are formulated as part of a cosmetically acceptable composition.

22. The method of claim 21, wherein the cosmetically acceptable composition further comprises one or more additional cosmetically acceptable ingredients.

23. The method of claim 22, wherein the one or more additional cosmetically acceptable ingredients are selected from amino acids, amino acid derivatives, peptides, vitamins, keratins, acidifiers, polycarboxylic acids, fatty acids, fatty alcohols, fatty acid esters, emulsifiers, emollients, gelling agents, antioxidants, oils, waxes, preservatives, sunscreens, and polyphenols.

24. The method of claim 1, wherein there is an increase in the denaturization temperature of the hair of 3° C. or more, 4° C. or more, or 5° C. or more following the application of the phytic acid or the combination of phytic acid and glucono-delta-lactone.

25. The method of claim 1, wherein there is an increase in the Young's Modulus of the hair of 3% or greater, 4% or greater, 5% or greater, 6% or greater, 7% or greater, 8% or greater, or 9% or greater following the application of the phytic acid or the combination of phytic acid and glucono-delta-lactone.

26. The method of claim 1, wherein there is an increase in the break stress of the hair of 3% or greater, 4% or greater, 5% or greater, 6% or greater, 7% or greater, 8% or greater, or 9% or greater following the application of the phytic acid or the combination of phytic acid and glucono-delta-lactone.

27. The method of claim 1, wherein there is an increase in the surface hydrophobicity of the human hair following the application of the phytic acid or the combination of phytic acid and glucono-delta-lactone.

28. The method of claim 1, wherein there is an increase in the water contact angle of the human hair as measured by differential wetting characterization (DWC) following the application of the phytic acid or the combination of phytic acid and glucono-delta-lactone.

29. The method of claim 1, wherein there is a reduction in protein loss of the hair following the application of the phytic acid or the combination of phytic acid and glucono-delta-lactone.

30. The method of claim 1, wherein there is greater than 25% reduction in the protein loss as measured by Modified Lowry Protein Assay following the application of the phytic acid or the combination of phytic acid and glucono-delta-lactone.
